(19) United States
(12) Patent Application Publication
(10) Pub. No.: US 2024/0374743 A1
(43) Pub. Date: Nov. 14, 2024

(54) BLETILLA STRIATA POLYSACCHARIDE IRON COMPLEX, PREPARATION METHOD THEREFOR AND USE THEREOF

(71) Applicant: Qingxiang GUAN, Changchun (CN)

(72) Inventor: Qingxiang GUAN, Changchun (CN)

(21) Appl. No.: 18/660,885

(22) Filed: May 10, 2024

(30) Foreign Application Priority Data
May 12, 2023 (CN) 2023105386159

(51) Int. Cl. A61K 47/61; A61P 7/06; C08B 37/00

(52) U.S. Cl. CPC A61K 47/61; A61P 7/06; C08B 37/0087

(57) ABSTRACT

A Bletilla striata polysaccharide-iron complex, a preparation method therefor and use thereof are disclosed. The preparation method comprises: dissolving Bletilla striata polysaccharide and sodium citrate in distilled water, and under stirring condition, dropwise adding an NaHCO₃ solution while heating to adjust a pH value of a reaction system; when a temperature of the reaction system rises to a reaction temperature, alternately dropwise adding the NaHCO₃ solution and an FeCl₃ solution to allow reaction until a red insoluble matter appears in the reaction system; and continuing the reaction to obtain a reaction solution; and subjecting the reaction solution to cooling, centrifugation, concentration, alcoholic precipitation, suction filtration, drying, redissolution, dialysis, and freezing to obtain the Bletilla striata polysaccharide-iron complex. The Bletilla striata polysaccharide-iron complex prepared in the present disclosure has a higher iron content and a significantly enhanced antioxidant ability.

### CROSS-REFERENCE TO RELATED APPLICATION

This application is filed on the basis of and claims the priority of Chinese Patent Application No. 202310538615.9 filed May 12, 2023, the entire contents of which are incorporated herein by reference.

### TECHNICAL FIELD

The present disclosure belongs to the technical field of polymeric polysaccharides, and in particular relates to a Bletilla striata polysaccharide-iron complex, a preparation method therefor and use thereof.

### BACKGROUND

Iron deficiency anemia is the most common nutritional deficiency disease in the world at present. Although traditional chemical drugs for treating iron deficiency anemia, such as ferrous sulfate and other ferrous ion preparations, have fast onset of action and good curative effect, free ferrous ions therefrom upon administration may result in free radicals in vivo, which cause cell membrane damage, leading to many adverse gastrointestinal reactions. Trivalent polysaccharide-iron preparations have the advantages of a stable structure, few side effects, a high bioavailability, a good efficacy, etc.

Polysaccharides extracted from plants, such as Chinese yam polysaccharide, corn polysaccharide, tea polysaccharide, ginseng polysaccharide, Codonopsis pilosula polysaccharide, Angelica sinensis polysaccharide, and Bletilla striata polysaccharide, not only have good water solubility and metal complexing ability, but also have intrinsically many pharmacological effects, such as antioxidant effect, anti-inflammatory and bacteriostatic effects, immunomodulatory effect, blood sugar and lipid regulating effects, etc., and also have certain therapeutic and preventive effects on diseases. Polysaccharide-iron complexes prepared by complexing such polysaccharides with ferric iron can eliminate side effects caused by free ferrous ions, improve the absorption and therapeutic effects of drugs, and function the intrinsic pharmacological effects of polysaccharides, thus becoming novel iron supplements.

At present, there has been no report about Bletilla striata polysaccharide-iron complexes.

### SUMMARY

The present disclosure aims at solving at least one of the above-mentioned technical problems existing in the prior art. To this end, the present disclosure provides a Bletilla striata polysaccharide-iron complex, a preparation method therefor and use thereof. The Bletilla striata polysaccharide-iron complex is prepared by fed-batch synthesis by selecting appropriate preparation raw materials and adjusting the preparation process. Compared with traditional preparation methods, the prepared Bletilla striata polysaccharide-iron complex has a higher iron content, a better iron supplementation effect, and a significantly enhanced antioxidant ability.

In order to solve the above-mentioned technical problems, a first aspect of the present disclosure provides a method for preparing a Bletilla striata polysaccharide-iron complex, comprising the following steps:
(1) dissolving Bletilla striata polysaccharide and sodium citrate in distilled water, and under stirring condition, dropwise adding an NaHCO₃ solution while heating to adjust a pH value of a reaction system; when a temperature of the reaction system rises to a reaction temperature, alternately dropwise adding the NaHCO₃ solution and an FeCl₃ solution to allow reaction until a red insoluble matter appears in the reaction system; and continuing the reaction to obtain a reaction solution; and
(2) subjecting the reaction solution to cooling, centrifugation, concentration, alcoholic precipitation, suction filtration, drying, redissolution, dialysis, and freezing to obtain the Bletilla striata polysaccharide-iron complex.

In particular, Bletilla striata polysaccharide is a main component of Bletilla striata, and is mainly composed of β-1,4-glucose residue, β-1,4-mannose residue and α-1,6-glucose residue bonded each other. Bletilla striata polysaccharide has pharmacological effects such as antioxidant, anti-inflammatory, antimicrobial, anti-ulcer, and wound healing-promoting effects, is cheap, easy to obtain, safe, and mild, and has a wide range of sources. In addition, Bletilla striata polysaccharide contains a large number of hydroxyl structures and can be complexed with iron ions; therefore, a Bletilla striata polysaccharide-iron complex is prepared from Bletilla striata polysaccharide and an FeCl₃ solution as raw materials in the present disclosure.

When preparing the Bletilla striata polysaccharide-iron complex of the present disclosure, NaHCO₃ as a pH regulator is more conducive to improving the iron content of the Bletilla striata polysaccharide-iron complex as compared with the traditional pH regulator NaOH. The reason is as follows: NaOH is a strongly alkaline solution, which easily causes a substantial change in the pH value of the solution system when it is dropwise added too much or too little during the process of adjusting the pH value of the solution, leading to the instability of the pH value of the solution, resulting in excess alkalinity, whereby the complexation of iron ions with polysaccharides is disturbed by too much alkali, leading to a decrease in the iron content; whereas NaHCO₃ is weakly alkaline in solution, is less sensitive to the pH of the solution, does not cause a significant change in the pH value of the solution, and is relatively stable in solution, and a stable pH value is more conducive to normal complexation of iron ions with polysaccharides, thus increasing the iron content of the Bletilla striata polysaccharide-iron complex.

Preferably, in step (1), the pH value is 7.0-11.5.

Preferably, in step (1), the reaction temperature is 50-90° C.

Preferably, in step (1), the reaction is continued for 0.5-3.0 hours.

Preferably, the Bletilla striata polysaccharide has a purity of 30%-99%, a mass ratio of the Bletilla striata polysaccharide to sodium citrate is (0.25-4): 1, and a mass-to-volume ratio of the Bletilla striata polysaccharide to the distilled water is (1-4) g: 100 mL.

Preferably, the NaHCO₃ solution has a mass concentration of 5%-30%, and the FeCl₃ solution has a molar concentration of 1-5 mol/L.

Preferably, a ratio of a dropwise addition rate of the NaHCO₃ solution to that of the FeCl₃ solution is 1:(3-5).

Preferably, in step (2), the cooling comprises cooling to room temperature under −80° C. to 4° C.

Preferably, in step (2), the centrifugation is conducted at a rotation speed of 1500-4000 r/min for 5-20 min.

Preferably, in step (2), the concentration comprises: collecting a supernatant resulting from the centrifugation, and subjecting the supernatant to rotary evaporation at 70-100° C. to ½-¼ of an original volume to obtain a concentrate.

Preferably, in step (2), the alcoholic precipitation comprises: adding an ethanol solution to the concentrate, stirring a resulting solution at 60-120 r/min for 3-5 min to fully precipitate out a precipitate, and leaving the solution to stand at room temperature for 12-36 h, wherein the ethanol solution has a volume concentration of 50%-95%, and a volume ratio of the ethanol solution to the concentrate is (1-4): 1.

Preferably, in step (2), the drying is conducted at a temperature of 50-90° C., for 4-12 h, whereby a dried product is obtained.

Preferably, in step (2), the redissolution comprises redissolving the dried product in distilled water, wherein a mass-to-volume ratio of the dried product to the distilled water is (1.5-10) g: 100 mL.

Preferably, in step (2), the dialysis is conducted by using a semi-permeable membrane with a molecular weight cut-off of 8000-14000 Da for desalting for 50-80 hours.

Preferably, in step (2), the freezing comprises: subjecting a solution after being subjected to dialysis to rotary evaporation at 70-100° C. to ½-⅕ of an original volume to obtain a rotary evaporation product, then prefreezing the rotary evaporation product at −20° C. to −80° C. for 1-7 days to obtain a prefreezed product, and then freeze-drying the prefreezed product in a vacuum for 1-5 days.

A second aspect of the present disclosure provides a Bletilla striata polysaccharide-iron complex. The Bletilla striata polysaccharide-iron complex is prepared by the method for preparing a Bletilla striata polysaccharide-iron complex according to the first aspect of the present disclosure, wherein the Bletilla striata polysaccharide-iron complex has an iron content of 17.57±0.569% and has a good iron supplementation effect.

A third aspect of the present disclosure provides an iron supplement preparation. The iron supplement preparation comprises the Bletilla striata polysaccharide-iron complex according to the second aspect of the present disclosure.

Compared with the prior art, the above technical solution of the present disclosure has at least the following technical effects or advantages.
(1) In the present disclosure, a Bletilla striata polysaccharide-iron complex is prepared by fed-batch synthesis from Bletilla striata polysaccharide and an FeCl₃ solution as main raw materials and NaHCO₃ as a pH regulator and by adjusting the preparation process and controlling the process parameters. Compared with traditional preparation methods, the prepared Bletilla striata polysaccharide-iron complex has a higher iron content and a significantly improved antioxidant ability as compared to Bletilla striata polysaccharide.
(2) The Bletilla striata polysaccharide-iron complex prepared by the present disclosure has an iron content of 17.57±0.569%, which can effectively improve various indexes of iron deficiency anemia, specifically including body weight recovery, hematological index restoration, serum iron supplementation, alleviation of pathologies in liver and spleen, etc.

### BRIEF DESCRIPTION OF DRAWINGS

FIG. 1 is a flow chart of the preparation process of Example 1.

FIG. 2 is an infrared spectrum of Bletilla striata polysaccharide and Bletilla striata polysaccharide-iron prepared in Example 1.

FIG. 3A is a hydrogen nuclear magnetic resonance spectrum of Bletilla striata polysaccharide; FIG. 3B is a hydrogen nuclear magnetic resonance spectrum of the Bletilla striata polysaccharide-iron complex prepared in Example 1; and FIG. 3C is a hydrogen nuclear magnetic resonance spectrum of a mixture of Bletilla striata polysaccharide and ferric trichloride.

FIG. 4 is an X-ray diffraction pattern of Bletilla striata polysaccharide and the Bletilla striata polysaccharide-iron complex prepared in Example 1.

FIG. 5A and FIG. 5B are scanning electron microscope (SEM) images of Bletilla striata polysaccharide with different magnifications; and FIG. 5C and FIG. 5D are SEM images of the Bletilla striata polysaccharide-iron complex prepared in Example 1 with different magnifications.

FIG. 6A is a thermogravimetric analysis diagram of Bletilla striata polysaccharide; and FIG. 6B is a thermogravimetric analysis diagram of the Bletilla striata polysaccharide-iron complex prepared in Example 1.

FIG. 7A and FIG. 7B are graphs showing the antioxidant properties of Bletilla striata polysaccharide and the Bletilla striata polysaccharide-iron complex prepared in Example 4 at a same concentration.

FIG. 8 is a diagram showing the effect of the Bletilla striata polysaccharide-iron complex prepared in Example 4 on the body weight of mice.

FIG. 9A, FIG. 9B and FIG. 9C are diagrams showing the effect of the Bletilla striata polysaccharide-iron complex prepared in Example 4 on hematological indexes in mice.

FIG. 10 is a diagram showing the effect of the Bletilla striata polysaccharide-iron complex prepared in Example 4 on serum iron in mice.

FIG. 11 is a diagram showing the effect of the Bletilla striata polysaccharide-iron complex prepared in Example 4 on unsaturated iron in serum iron in mice.

FIG. 12A and FIG. 12B are diagrams showing the effect of the Bletilla striata polysaccharide-iron complex prepared in Example 4 on the organ coefficients of spleen and liver in mice.

### DETAILED DESCRIPTION

The present disclosure is described below in detail in conjunction with examples, so as to facilitate those skilled in the pertinent art to understand the present disclosure. It is necessary to point out in particular herein that the examples are only used to further illustrate the present disclosure and cannot be construed as limiting the scope of protection of the present disclosure. Non-essential modifications and adjustments made by those skilled in the art according to the above summary should still fall within the scope of protection of the present disclosure. In addition, the raw materials mentioned below that are not specified in detail are all commercial products; and the process steps or preparation methods that are not mentioned in detail are all process steps or preparation methods known to those skilled in the art.

### Example 1

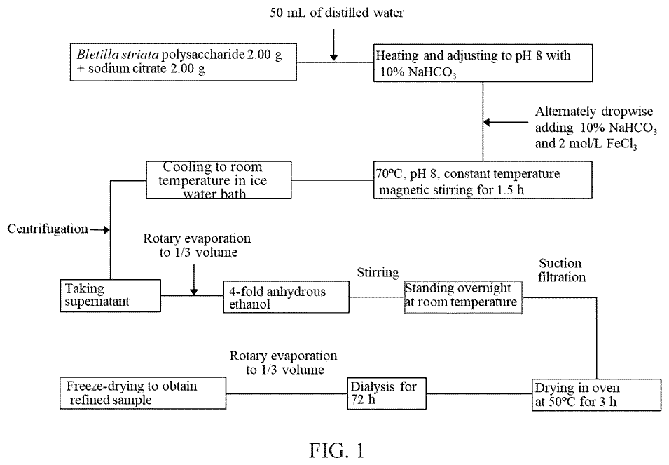

A method for preparing a Bletilla striata polysaccharide-iron complex was provided, the flow chart of the preparation process of which was shown in FIG. 1. The specific steps of the method were as follows:
(1) 2 g of Bletilla striata polysaccharide and 2 g of sodium citrate powder were weighed and dissolved in 50 mL of distilled water; the solution was then poured into a magnetic stirrer with constant temperature heating, and a pH value of the reaction system was adjusted to 8 with a NaHCO₃ solution with a mass concentration of 10% while heating; and when the temperature reached 70° C., the NaHCO₃ solution with a mass concentration of 10% and 2 mol/L FeCl₃ were alternately added dropwise until a red insoluble matter appeared in the reaction system, and the reaction was continued for 1.5 hours to obtain a reaction solution; and
(2) the reaction solution prepared in step (1) was cooled to room temperature with a cold water bath at 0° C. and centrifuged at a rotation speed of 4000 r/min for 10 min, and a supernatant was collected and concentrated to ⅓ of an original volume by rotary evaporation at 90° C. to obtain a concentrate; 75% ethanol solution, which was four times as much as the concentrate, was added to the concentrate and stirred for 4 min at a rotation speed of 110 r/min to fully precipitate out a precipitate, and the solution was then left to stand at room temperature for 20 h; suction filtration in a vacuum was carried out with a qualitative filter paper, and the obtained filter cake was put into a crucible and dried in an oven at 50° C. for 6 h to obtain a dried product; the dried product was redissolved in 70 mL of distilled water and subjected to dialysis with 10000 Da semi-permeable membrane for desalting for 72 hours to obtain a dialysate; and the dialysate was concentrated at 90° C. to ⅓ of an original volume, then pre-frozen in a refrigerator at −20° C. for 3 days, and then freeze-dried in a vacuum freeze-dryer for 3 days to obtain a sample of the Bletilla striata polysaccharide-iron complex of this example.

### Example 2

A method for preparing a Bletilla striata polysaccharide-iron complex was provided, comprising the following steps:
(1) 2 g of Bletilla striata polysaccharide and 1 g of sodium citrate powder were weighed and dissolved in 50 mL of distilled water; the solution was then poured into a magnetic stirrer with constant temperature heating, and a pH value of the reaction system was adjusted to 8.5 with a NaHCO₃ solution with a mass concentration of 10% while heating; and when the temperature reached 80° C., the NaHCO₃ solution with a mass concentration of 10% and 2 mol/L FeCl₃ were alternately added dropwise until a red insoluble matter appeared in the reaction system, and the reaction was continued for 1.5 hours to obtain a reaction solution; and
(2) the reaction solution prepared in step (1) was cooled to room temperature with a cold water bath at 0° C. and centrifuged at a rotation speed of 4000 r/min for 15 min, and a supernatant was collected and concentrated to ⅓ of an original volume by rotary evaporation at 90° C. to obtain a concentrate; 50% ethanol solution, which was four times as much as the concentrate, was added to the concentrate and stirred for 4 min at a rotation speed of 110 r/min to fully precipitate a precipitate, and the solution was then left to stand at room temperature for 20 h; suction filtration in a vacuum was carried out with a qualitative filter paper, and the obtained filter cake was put into a crucible and dried in an oven at 50° C. for 10 h to obtain a dried product; the dried product was redissolved in 80 mL of distilled water and subjected to dialysis with 10000 Da semi-permeable membrane for desalting for 72 hours to obtain a dialysate; and the dialysate was concentrated at 90° C. to ⅓ of an original volume, then pre-frozen in a refrigerator at −20° C. for 3 days, and then freeze-dried in a vacuum freeze-dryer for 3 days to obtain a sample of the Bletilla striata polysaccharide-iron complex of this example.

### Example 3

A method for preparing a Bletilla striata polysaccharide-iron complex was provided, comprising the following steps:
(1) 2 g of Bletilla striata polysaccharide and 0.5 g of sodium citrate powder were weighed and dissolved in 50 mL of distilled water; the solution was then poured into a magnetic stirrer with constant temperature heating, and a pH value of the reaction system was adjusted to 9 with a NaHCO₃ solution with a mass concentration of 10% while heating; and when the temperature reached 90° C., the NaHCO₃ solution with a mass concentration of 10% and 2 mol/L FeCl₃ were alternately added dropwise until a red insoluble matter appeared in the reaction system, and the reaction was continued for 1.5 hours to obtain a reaction solution; and
(2) the reaction solution prepared in step (1) was cooled to room temperature with a cold water bath at 4° C. and centrifuged at a rotation speed of 3500 r/min for 15 min, and a supernatant was collected and concentrated to ⅓ of an original volume by rotary evaporation at 90° C. to obtain a concentrate; 75% ethanol solution, which was four times as much as the concentrate, was added to the concentrate and stirred for 4 min at a rotation speed of 110 r/min to fully precipitate a precipitate, and the solution was then left to stand at room temperature for 20 h; suction filtration in a vacuum was carried out with a qualitative filter paper, and the obtained filter cake was put into a crucible and dried in an oven at 50° C. for 10 h to obtain a dried product; the dried product was redissolved in 100 mL of distilled water and subjected to dialysis with 10000 Da semi-permeable membrane for desalting for 72 hours to obtain a dialysate; and the dialysate was concentrated at 90° C. to ⅓ of an original volume, then pre-frozen in a refrigerator at −20° C. for 3 days, and then freeze-dried in a vacuum freeze-dryer for 3 days to obtain a sample of the Bletilla striata polysaccharide-iron complex of this example.

### Example 4

A method for preparing a Bletilla striata polysaccharide-iron complex was provided, comprising the following steps:
(1) 1.35 g of Bletilla striata polysaccharide and 1 g of sodium citrate powder were weighed and dissolved in 50 mL of distilled water; the solution was then poured into a magnetic stirrer with constant temperature heating, and a pH value of the reaction system was adjusted to 9.2 with a NaHCO₃ solution with a mass concentration of 10% while heating; and when the temperature reached 81° C., the NaHCO₃ solution with a mass concentration of 10% and 2 mol/L FeCl₃ were alternately added dropwise until a red insoluble matter appeared in the reaction system, and the reaction was continued for 1.3 hours to obtain a reaction solution; and
(2) the reaction solution prepared in step (1) was cooled to room temperature with a cold water bath at 0° C. and centrifuged at a rotation speed of 4000 r/min for 15 min, and a supernatant was collected and concentrated to ⅓ of an original volume by rotary evaporation at 90° C. to obtain a concentrate; 75% ethanol solution, which was four times as much as the concentrate, was added to the concentrate and stirred for 4 min at a rotation speed of 110 r/min to fully precipitate a precipitate, and the solution was then left to stand at room temperature for 20 h; suction filtration in a vacuum was carried out with a qualitative filter paper, and the obtained filter cake was put into a crucible and dried in an oven at 50° C. for 10 h to obtain a dried product; the dried product was redissolved in 70 mL of distilled water and subjected to dialysis with 10000 Da semi-permeable membrane for desalting for 72 hours to obtain a dialysate; and the dialysate was concentrated at 90° C. to ⅓ of an original volume, then pre-frozen in a refrigerator at −20° C. for 3 days, and then freeze-dried in a vacuum freeze-dryer for 3 days to obtain a sample of the Bletilla striata polysaccharide-iron complex of this example.

### Comparative Example 1

The only difference between Comparative Example 1 and Example 1 was that when preparing the Bletilla striata polysaccharide-iron complex of Comparative Example 1, the NaHCO₃ solution in Example 1 was replaced by a NaOH solution having a same concentration, and the types and amounts of the other raw materials, the preparation steps, and the process parameters were all the same as those in Example 1.

### Comparative Example 2

In Comparative Example 2, a Bletilla striata polysaccharide-iron complex was prepared by co-thermal synthesis, and the specific steps were as follows:
(1) 2 g of Bletilla striata polysaccharide and 2 g of sodium citrate powder were weighed and dissolved in 50 mL of distilled water, and a pH of the solution was adjusted to 8 with a NaHCO₃ solution with a mass concentration of 10%; and then, 5 mL of 2 mol/L FeCl₃ solution and 15 mL of the NaHCO₃ solution with a mass concentration of 10% were poured into a stoppered conical flask at 70° C. under magnetic stirring, and reacted for 2 h to obtain a reaction solution; and
(2) the reaction solution prepared in step (1) was cooled to room temperature with a cold water bath at 0° C. and centrifuged at a rotation speed of 4000 r/min for 10 min, and a supernatant was collected and concentrated to ⅓ of an original volume by rotary evaporation at 90° C. to obtain a concentrate; 75% ethanol solution, which was four times as much as the concentrate, was added to the concentrate and stirred for 4 min at a rotation speed of 110 r/min to fully precipitate out a precipitate, and the solution was then left to stand at room temperature for 20 h; suction filtration in a vacuum was carried out with a qualitative filter paper, and the obtained filter cake was put into a crucible and dried in an oven at 50° C. for 6 h to obtain a dried product; the dried product was redissolved in 70 mL of distilled water and subjected to dialysis with 10000 Da semi-permeable membrane for desalting for 72 hours to obtain a dialysate; and the dialysate was concentrated at 90° C. to ⅓ of an original volume, then pre-frozen in a refrigerator at −20° C. for 3 days, and then freeze-dried in a vacuum freeze-dryer for 3 days to obtain a sample of the Bletilla striata polysaccharide-iron complex of this comparative example.

### Performance Test

### 1. Iron Content

The Bletilla striata polysaccharide-iron complexes prepared in Example 1 and Comparative Examples 1 and 2 were tested for iron content, and the results were shown in Table 1.

TABLE 1

| | | | Co-thermal |
|---|---|---|---|
| | Fed-batch synthesis | | synthesis |
| Iron binding | Comparative | | Comparative |
| rate (%) | Example 1 | Example 1 | Example 2 |
| 1 | 14.3 | 17.4 | 10.3 |
| 2 | 15.1 | 17.1 | 10.5 |
| 3 | 14.5 | 18.2 | 10.7 |
| X̅ ± SD | 14.63 ± 0.416 | 17.57 ± 0.569 | 10.50 ± 0.200 |
| RSD (%) | 2.84 | 3.23 | 1.90 |

As can be seen from Table 1, the iron content of the Bletilla striata polysaccharide-iron complex prepared in Example 1 has an average value (X̅±SD) of 17.57±0.569% after three tests, with a relative standard deviation (RSD) of 3.23%; whereas the Bletilla striata polysaccharide-iron complex of Comparative Example 1, which was prepared by using a NaOH solution as a pH value regulator, has an iron content of 14.63±0.416%, and the Bletilla striata polysaccharide-iron complex of Comparative Example 2, which was prepared by using the co-thermal method, has an iron content of 10.50±0.200%. That is, the iron content of the Bletilla striata polysaccharide-iron complex prepared by the preparation method of the present disclosure is higher than that of Bletilla striata polysaccharide-iron complex prepared by the conventional co-thermal synthesis method and that of Bletilla striata polysaccharide-iron complex prepared by the fed-batch synthesis method with NaOH solution as a pH value regulator.

### 2. Infrared Spectroscopy

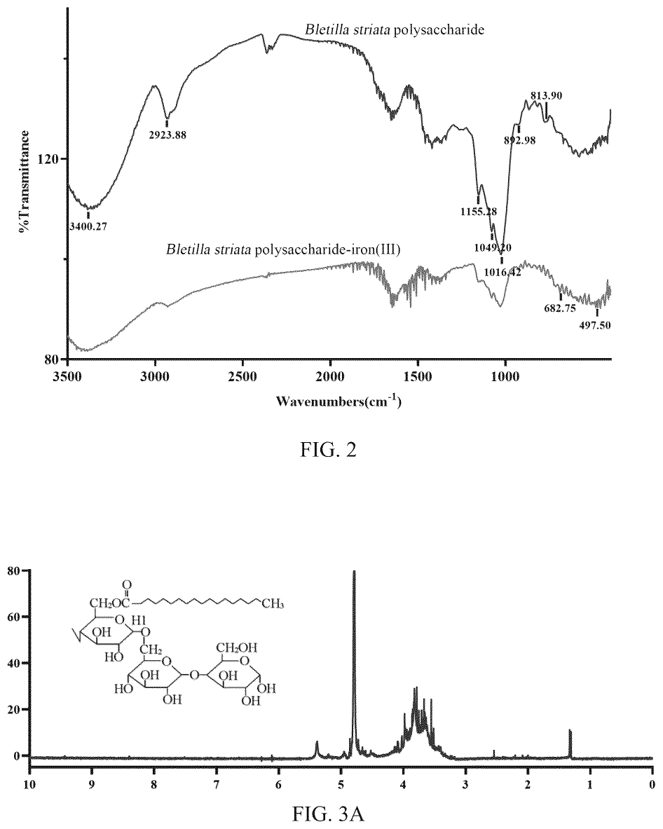

As could be seen from the infrared spectrum of Bletilla striata polysaccharide in FIG. 2: the Bletilla striata polysaccharide has a strong and broad absorption peak at 3400.27 cm⁻¹, which corresponds to its hydroxyl structure and is the most obvious peak; the Bletilla striata polysaccharide has an absorption peak at 2923.88 cm⁻¹, which corresponds to an asymmetric C—H stretching vibration in its structure; the Bletilla striata polysaccharide has absorption peaks at 892.98 cm⁻¹ and 813.90 cm⁻¹, which are the B-glucose residue and mannose residue in its structure; and a strong absorption peak at 1000-1200 cm⁻¹ represents a characteristic peak of a pyran ring structure in Bletilla striata polysaccharide. In addition, as can be seen from the infrared spectrum of Bletilla striata polysaccharide-iron (III) in FIG. 2: a significant change of the characteristic peak can be seen intuitively, and the original strong and wide characteristic peak is weakened and shifted backward; there are a strong absorption peak with sensitive change at 400-900 cm⁻¹ and characteristic absorption peaks of iron core β-FeOOH at 682.75 cm⁻¹ and 497.60 cm⁻¹, indicating that a Bletilla striata polysaccharide-iron complex has been synthesized.

### 3. Hydrogen Nuclear Magnetic Resonance Spectrometry

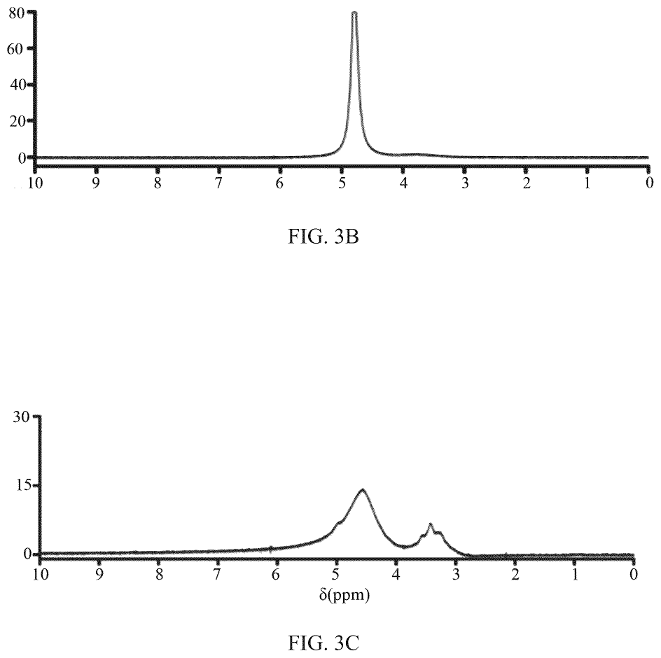

After comparing the hydrogen nuclear magnetic resonance spectrum of Bletilla striata polysaccharide in FIG. 3A with the hydrogen nuclear magnetic resonance spectrum of Bletilla striata polysaccharide-iron (III) complex in FIG. 3B, it can be seen that in addition to a solvent heavy water peak at δ 4.79 ppm, the original structural characteristic peak of the polysaccharide disappears due to the synthesis of the iron complex. The reason for this phenomenon may be that iron is paramagnetic, which leads to the amplification of the nuclear magnetic signal nearby, causing interference with the magnetic environment near the polysaccharide, lengthening the relaxation time of the other absorption peaks of the ligand polysaccharide, which are visible, broadening the spectral peak, and thus causing apparent nuclear magnetic invisibility. In order to demonstrate that the disappearance of the characteristic peak is truly due to the synthesis of the Bletilla striata polysaccharide-iron (III) complex, a simple physical mixture of Bletilla striata polysaccharide and ferric chloride was detected in the present disclosure to obtain a hydrogen nuclear magnetic resonance spectrum. From the hydrogen nuclear magnetic resonance spectrum of the physical mixture of Bletilla striata polysaccharide and ferric chloride in FIG. 3C, it could be seen that the nuclear magnetic resonance spectrum of the simple physical mixture is significantly different from that of the synthesized Bletilla striata polysaccharide-iron complex, thus proving that a Bletilla striata polysaccharide-iron (III) complex, rather than a simple physical mixture, is synthesized in the present disclosure.

### 4. X-Ray Diffraction

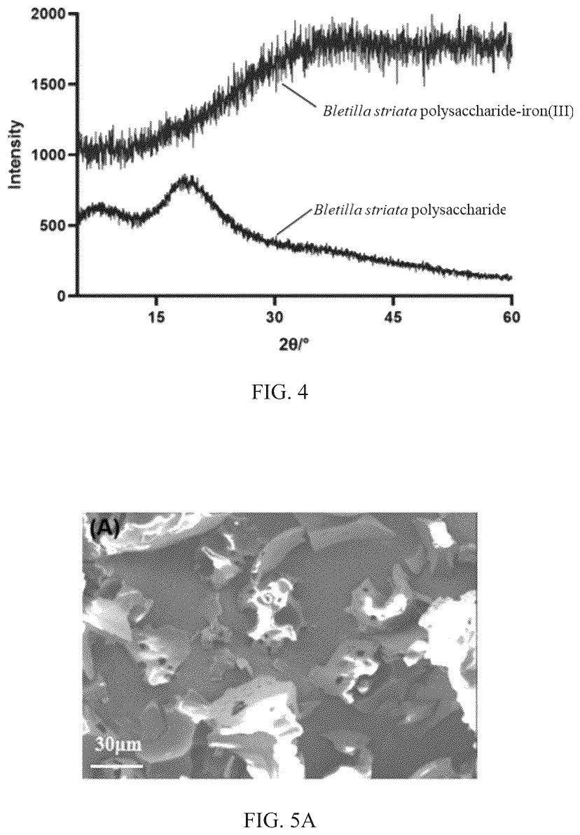

It can be seen from FIG. 4 that the X-Ray Diffraction (XRD) pattern of Bletilla striata polysaccharide has weaker characteristic peaks at 2θ of 7.826° and 19.331°, and these weak peaks are both in amorphous regions, indicating that the degree of crystallization of Bletilla striata polysaccharide is not high and the Bletilla striata polysaccharide has a good water solubility. Whereas the peak shape in the XRD pattern of the Bletilla striata polysaccharide-iron complex has changed greatly as compared with that of Bletilla striata polysaccharide, wherein there are two extremely weak peaks at 2θ of 35.376° and 61.195°, and no obvious characteristic peak is found. It can thus be seen that the Bletilla striata polysaccharide-iron complex has an amorphous structure.

### 5. Scanning Electron Microscopy

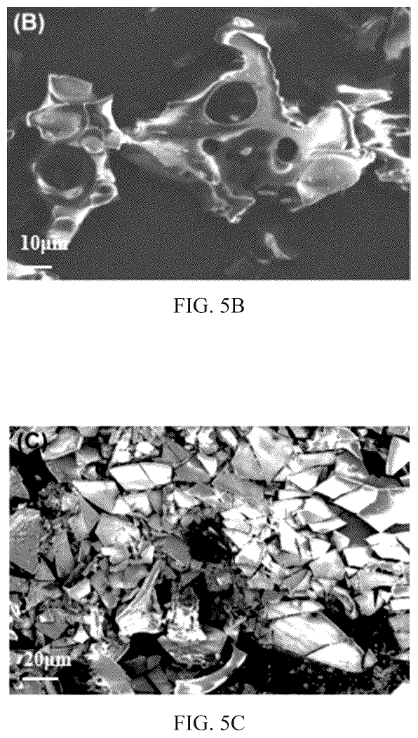

FIG. 5A and FIG. 5B were SEM images of Bletilla striata polysaccharide. As can be seen from FIG. 5A and FIG. 5B, Bletilla striata polysaccharide is in an adhesive state, has voids in the shape of round pores in its structure, and an uneven surface. FIG. 5C and FIG. 5D are SEM images of the Bletilla striata polysaccharide-iron complex. As can be seen from FIG. 5C and FIG. 5D, compared with the Bletilla striata polysaccharide, the structure of the Bletilla striata polysaccharide-iron complex has significantly changed both in shape and size, the surface of the Bletilla striata polysaccharide-iron shows no adhesion and has a smooth sheet-like structure, and the particle size of the Bletilla striata polysaccharide-iron complex molecules is smaller than that the Bletilla striata polysaccharide, which is due to the fact that when Fe³⁺ and the polysaccharide are coordinated and complexed with each other, the molecular conformation and degree of cross-linking are changed, which leads to the change of the surface thereof from porous to smooth and from irregularly shape to relatively regular sheet-like.

### 6. Thermogravimetric Analysis

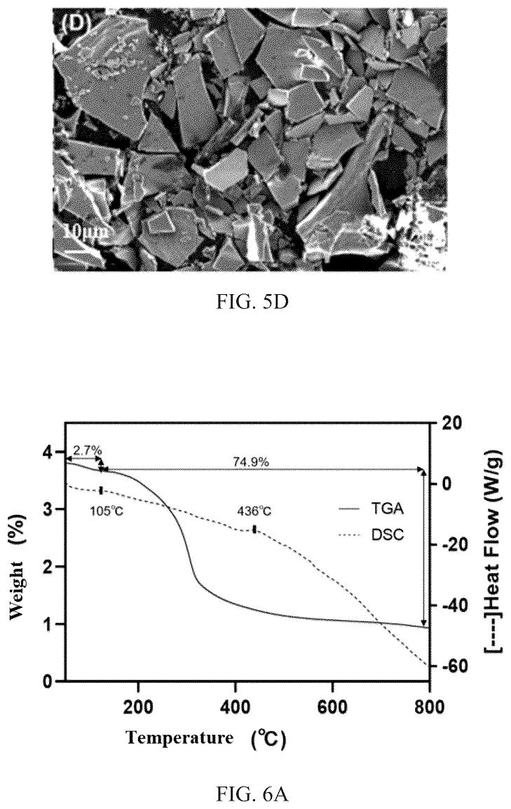

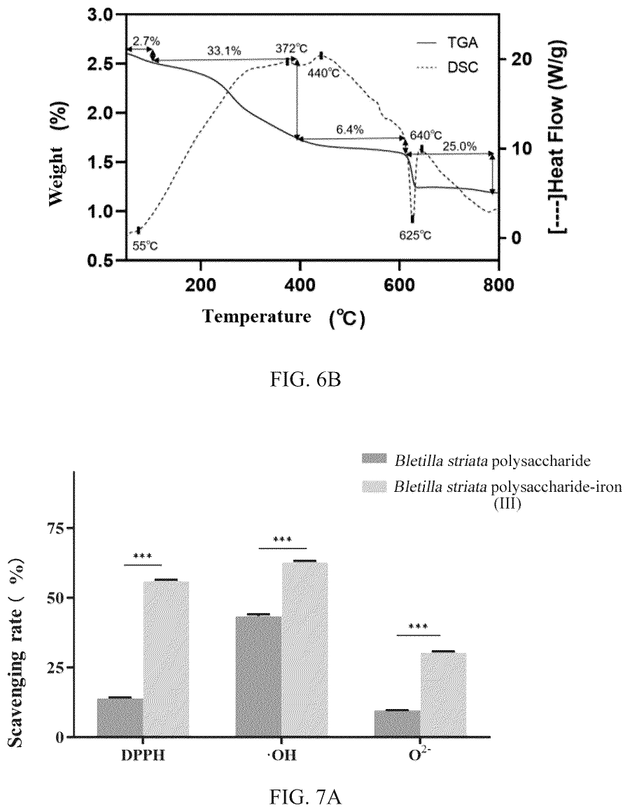

FIG. 6A and FIG. 6B are thermogravimetric analysis diagrams of Bletilla striata polysaccharide and the Bletilla striata polysaccharide-iron complex prepared in Example 1, wherein a Differential Scanning calorimeter (DSC) curve shows the change of thermodynamic parameters (such as melting endotherm and crystallization exotherm) as the phase state of a substance changes, while a Thermo Gravimetric Analysis (TGA) curve shows the weight loss of the substance as the programmed temperature changes. FIG. 6A is a TGA/DSC curve of Bletilla striata polysaccharide. In the DSC curve of FIG. 6A, an endothermic peak at about 105° C. is due to the sublimation of water, which is consistent with the TGA curve; after that, the DSC curve shows a downward trend, indicating that the polysaccharide is in a continuous endothermic process, with slight exotherm appearing at 436° C. and then endothermic decomposition. In the TGA curve of FIG. 6A, a first part of weight loss is at about 105° C., which is caused by the sublimation of adsorbed water and bound water in the polysaccharide polymer, with a weight loss rate of 2.7%; and a second part of weight loss was is temperatures of 205° C. and 322° C., respectively, which is caused by the thermal decomposition of the polysaccharide, with a weight loss rate of 74.9%. FIG. 6B is a TGA/DSC curve of Bletilla striata polysaccharide-iron complex. As can be seen from FIG. 6B, there are two endothermic peaks and three exothermic peaks in the DSC curve, wherein the endothermic peak at 73° C. is caused by the sublimation of water in the polysaccharide, and the two small and wide exothermic peaks at 372° C. and 440° C. are highly correlated with the two instances of weight loss at 228° C. and 421° C. in the TGA curve. In this regard, the Bletilla striata polysaccharide-iron complex not only has a loss of crystal water in the polysaccharide and a weight loss after decomposition, but also has a process of breakage between other bonds and the iron core bonds, and phase transformation, accompanied by continuous and complicated endothermic and exothermic processes. It is thus preliminarily judged that the melting point of the Bletilla striata polysaccharide-iron complex may be at about 372-440° C., indicating that the Bletilla striata polysaccharide-iron complex not only has good thermal stability, but also indirectly shows that the Bletilla striata polysaccharide-iron complex has a degree of polymerization which is not high and a good solubility. There is a strong endothermic peak at 625° C., followed by immediately an exothermic peak at 640° C., at which point complete thermal decomposition and weight loss of the polysaccharide and iron core may occur. The weight loss rates of the four parts in the TGA curve are 2.7%, 33.1%, 6.4%, and 25.0%, respectively. As can be seen from FIG. 6A and FIG. 6B, the thermogravimetric curve of the Bletilla striata polysaccharide-iron complex is smoother than that of Bletilla striata polysaccharide, indicating that the iron-modified Bletilla striata polysaccharide shows a better thermal stability.

### 7. Antioxidant Experiment In Vitro

### (1) Experimental Process

The sample used in this experiment is the Bletilla striata polysaccharide-iron complex prepared in Example 4.

### 1) DPPH Free Radical Scavenging Rate

2 mL of a sample solution and 2 mL of 25 mg/L DPPH solution (1.25 mg of DPPH powder dissolved in 50 mL of anhydrous ethanol) were placed in a 10 mL glass test tube, mixed by vortex for 15 s, and then incubated at room temperature (25° C.) for 30 min. 50% ethanol solution was used for zeroing, and the absorbance was measured at 517 nm (n=3). A mixed solution of 2 mL of anhydrous ethanol, instead of the sample solution, and 2 mL of DPPH was used as a control group, and a mixed solution of 2 mL of the sample solution and 2 mL of anhydrous ethanol was used as a blank solution. The DPPH scavenging rate of the sample was calculated according to the following equation:

$$DPPH free radical scavenging rate ( \% ) = [ 1 - ( {A}_{s} - {A}_{0} ) / {A}_{c} ] \times 100 \qquad ( 1 )$$

in which Aₛ represents the absorbance value of the mixed solution of the sample and DPPH; A₀ represents the absorbance value of the blank solution; and A_{c} represents the absorbance value of the solution of the control group.

### 2) ABTS Free Radical Scavenging Rate

Establishment of standard curve: vitamin C (Vc) solutions with a series of concentrations (20, 40, 60, 80, and 100 μg/mL) were prepared with deionized water. 100 μL of these Vc solutions with different concentrations were mixed with 3 mL of an ABTS free radical working solution, the resulting mixed solutions were reacted for 6 min in the dark, and then tested for absorbance value at 734 nm. With the concentrations of the Vc solutions as abscissa and the absorbance as ordinate, a standard curve was plotted.

100 μL of a sample solution was added to 3 mL of an ABTS free radical working solution, and mixed uniformly by vortex for 15 s. The resulting solution was allowed for reaction at room temperature (25° C.) in the dark for 6 min, and then tested for absorbance value at 734 nm (n=3) after using the deionized water for zeroing. A mixed solution of 100 μL of deionized water, instead of the sample solution, and 3 mL of ABTS was used as a control group, and a mixed solution of 100 μL of the sample solution and 3 mL of deionized water was used as a blank solution. The ABTS scavenging rate of the sample was calculated according to the following equation:

$$ABTS free radical scavenging rate ( \% ) = [ 1 - ( {A}_{s} - {A}_{0} ) / {A}_{c} ] \times 100 \qquad ( 2 )$$

in which Aₛ represents the absorbance value of the mixed solution of the sample and ABTS; A₀ represents the absorbance value of the blank solution; and A_{c} represents the absorbance value of the solution of the control group.

### 3) Hydroxyl (OH) Free Radical Scavenging Rate

1 mL of a sample solution was taken, added with 1 mL of 6 mM FeSO₄ solution, 0.5 mL of 2 mM salicylic acid solution, and 1.0 mL of 6 mM H₂O₂ solution successively, and reacted for 30 min in a water bath at 37° C. After the reaction was completed, the absorbance value of the reaction solution was determined at 510 nm (n=3). A mixed solution in which 1 mL of deionized water was used instead of the sample solution was used as a control group, and a mixed solution in which 1 mL of deionized water was used instead of H₂O₂ solution was used as a blank solution. The hydroxyl (·OH) free radical scavenging rate of the sample was calculated according to the following equation:

$$( \cdot OH ) scavenging rate ( \% ) = [ 1 - ( {A}_{s} - {A}_{0} ) / {A}_{c} ] \times 100 \qquad ( 3 )$$

in which Aₛ represents the absorbance value of the mixed solution of the sample and H₂O₂; A₀ represents the absorbance value of the blank solution; and A_{c} represents the absorbance value of the solution of the control group.

### 4) Superoxide Anion (O²⁻) Free Radical Scavenging Rate

1 mL of deionized water was added to 2 mL of 50 mM Tris-HCl buffer (pH 8.2), mixed uniformly, and incubated at room temperature for 20 min. 2.9 mL of the above solution was taken, added with 1 mL of a sample solution and 0.1 mL of 6 mM pyrogallol solution, mixed uniformly, and reacted at room temperature for 5 min. Three drops of 10 mM HCl solution were dropped to the mixed solution to terminate the reaction, and the absorbance value thereof was determined at 320 nm (n=3). A mixed solution in which 1 mL of deionized water was used instead of the sample solution was used as a control group, and a mixed solution in which 0.1 mL of deionized water was used instead of pyrogallol solution was used as a blank solution. The superoxide anion (O²⁻) scavenging rate of the sample was calculated according to the following equation:

$$( {O}^{2 -} ) scavenging rate ( \% ) = [ 1 - ( {A}_{s} - {A}_{0} ) / {A}_{c} ] \times 100 \qquad ( 4 )$$

in which Aₛ represents the absorbance value of the mixed solution of the sample and pyrogallol; A₀ represents the absorbance value of the blank solution; and A_{c} represents the absorbance value of the solution of the control group.

(2) Antioxidant Ability of Bletilla striata Polysaccharide-Iron Complex

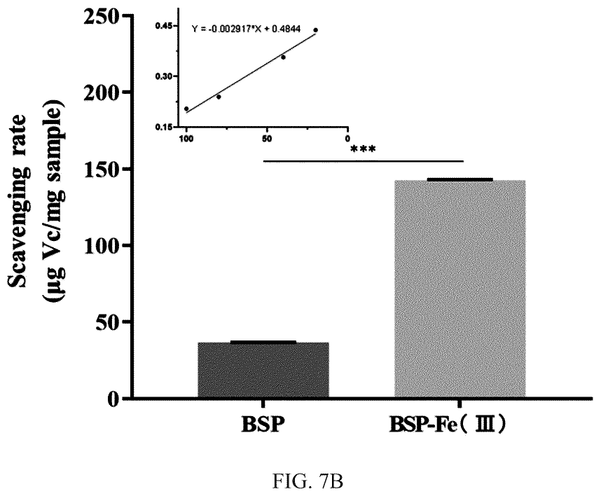

FIG. 7A and FIG. 7B respectively show the abilities of the Bletilla striata polysaccharide and the Bletilla striata polysaccharide-iron (III) complex at the same concentration to scavenge the four free radicals DPPH, ABTS, ·OH, and O²⁻, wherein the inset in FIG. 7B is a standard curve of Vc. It can be seen from FIG. 7A that the ability of the Bletilla striata polysaccharide-iron (III) complex to scavenge DPPH, ·OH, and O²⁻ is significantly higher than that of the Bletilla striata polysaccharide, and the free radical scavenging abilities are different by 42.13%, 19.46%, and 20.83%, respectively, with significant differences (p<0.001). It can be seen from FIG. 7B that at the same concentration, the scavenging rate of the Bletilla striata polysaccharide-iron (III) complex against ABTS free radicals is much higher than that of the Bletilla striata polysaccharide, and the scavenging rates thereof are equivalent to 36.81 and 142.77 μg of Vc per mg of sample, respectively, with significant differences (p<0.001). As can be seen from FIG. 7A and FIG. 7B, the Bletilla striata polysaccharide-iron (III) complex prepared by the present disclosure exhibits a good scavenging ability against the above four selected free radicals. It is demonstrated that after complexation with iron, the Bletilla striata polysaccharide does not lose its antioxidant ability despite the structural change, instead, the antioxidant ability thereof is increased significantly. This is because the complexation with iron changes the spatial structure of the original polysaccharide, which leads to the change of the electronic environment, so the antioxidant ability thereof is significantly increased.

### 8. Anti-Iron Deficiency Anemia Test in Mice

Experimental animals: 35 ICR mice, clean grade, body weight 20-25 g, female.

### (1) Experimental Process

Establishment of a mouse model of iron deficiency anemia: All mice were kept in a stainless steel cage, in which wood dust was used as padding to prevent iron from other sources from affecting the experimental results. The mice were adaptively fed at room temperature (24±1° C.) and humidity 50% for 3 days and then randomly divided into 7 groups, with 5 mice in each group. Five mice fed with basal feed (Fe>100 mg kg⁻¹) were used as a blank control group, and the remaining 30 mice were given a low-iron diet (Fe: 10 mg·kg⁻¹, AIN93 standard) and randomly divided into a model group (Model), a positive drug (Niferex) group, a ferrous sulfate (FeSO₄) group, and low-dose, medium-dose and high-dose groups of the Bletilla striata polysaccharide-iron (III) complex prepared in Example 4. After adaptive feeding, 0.1 mL of blood was taken from the cheeks of all mice, and the routine blood test data, serum iron three-term test data, and other data were determined by using a blood cell analyzer and used as normal values before modeling. Blood was drawn from the cheek of each mouse twice a week, but the blood was not collected. The mice in each group got free access to water. After three consecutive weeks of feeding, 0.2 mL of blood was collected from the cheeks of the mice in each group and then measured for various hematological indexes, and the body weight changes were recorded and plotted. When the hemoglobin content (Hb) value in red blood cells of mice decreased to less than 110 g/L, all the indexes decreased significantly, and the total iron binding capacity (TIBC) increased significantly, successful modeling was achieved. Mice that failed in modeling were excluded and were not included in any drug group.

Method of administration: Mice were administered by gavage at 8 am every day for 28 consecutive days. During gavage, the mice in each group got free access to water and fed normal diet. The mice in the blank control group and the model control group were given the same volume of distilled water, the mice in the positive drug group were given a drug with an Fe content of 0.039 mg/kg, the mice in FeSO₄ group were given a drug with an Fe content of 0.039 mg/kg, and the mice in the low-dose, medium-dose and high-dose groups of the Bletilla striata polysaccharide-iron (III) complex were given drugs with Fe contents of 0.0195 mg/kg, 0.039 mg/kg, and 0.078 mg/kg, respectively. Except the blank control group, the other six groups were given a low-iron feed. The body weight was weighed, recorded, and plotted once a week, and the animal state was observed.

Sample collection: After the last administration by gavage, the mice in each group got free access to water for 12 hours and sacrificed, whole blood was collected from the heart to determine various biochemical indexes, including hemoglobin content (Hb) in red blood cells, red blood cell count (RBC), hematocrit (HCT), mean corpuscular volume (MCV), mean corpuscular hemoglobin content (MCH), mean corpuscular hemoglobin concentration (MCHC), red blood cell volume distribution width (RDW), serum iron (SI) content, total iron binding capacity (TIBC), and erythropoietin (EPO). Liver and spleen tissues were taken, weighed, and recorded, and the organ index was calculated. The organ coefficient was calculated according to the following equation, and a histogram was plotted for analysis:

$$Organ coefficient ( g / 100 g ) = organ weight / animal body mass \times 100.$$

(2) Effect of Bletilla striata Polysaccharide-Iron Complex on the Body Weight of Mice

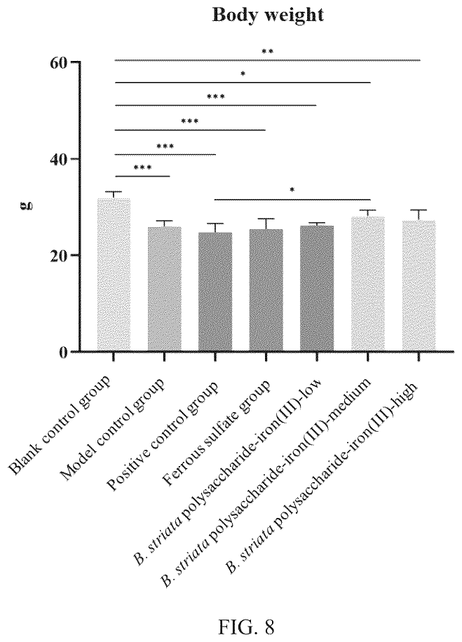

Iron deficiency anemia (IDA) makes patients lose their appetite and in turn causes weight loss or slow gain. As can be seen from FIG. 8, the weight gain of the mice in the IDA model group is significantly lower than that in the control group and drug groups, showing that the mice in all the drug groups has gained weight to varying extents after administration, indicating that the treatment with the drug could truly improve the problem of lack of appetite and gradually restore the ability of the body to transport nutrients normally. Although the 28-day administration by gavage can not achieve a long-term effect, the weight gains of the mice after 28 days of administration all tend to catch up with the control group. Among the groups, the mice in the Bletilla striata polysaccharide-iron (III) complex medium-dose group has the smallest significant difference from the mice in the control group (0.01<p<0.05), and the average body weight difference is 3.825 g (n=5); in addition, the average body weight of the mice in the Bletilla striata polysaccharide-iron (III) complex medium-dose group is significantly higher than the body weight of the mice in the Niferex group (0.01<p<0.05). The dose of the Bletilla striata polysaccharide-iron (III) complex medium-dose group is the same as that of the Niferex group, which indicates that at the same dose, the effect of the Bletilla striata polysaccharide-iron (III) complex on the weight gain of mice is superior to that of Niferex. The body weight of the mice in the FeSO₄ group is extremely significantly different from that in the control group, which also indicates that FeSO₄ does not perform well in improving the appetite problem of the body, so it is necessary to find an alternative drug to improve the problems caused thereby.

(3) Effect of Bletilla striata Polysaccharide-Iron Complex on the Haematological Indexes of Mice

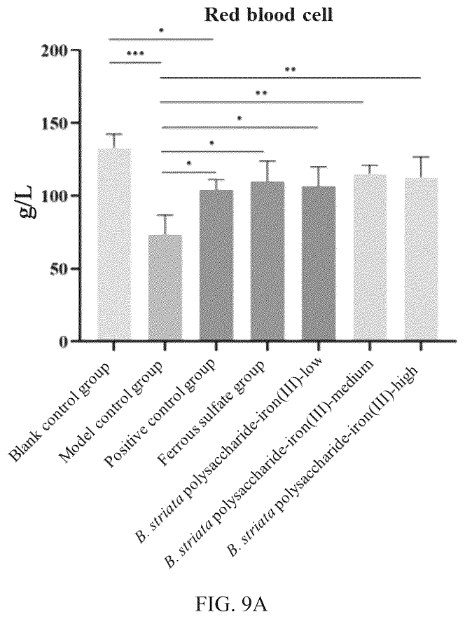

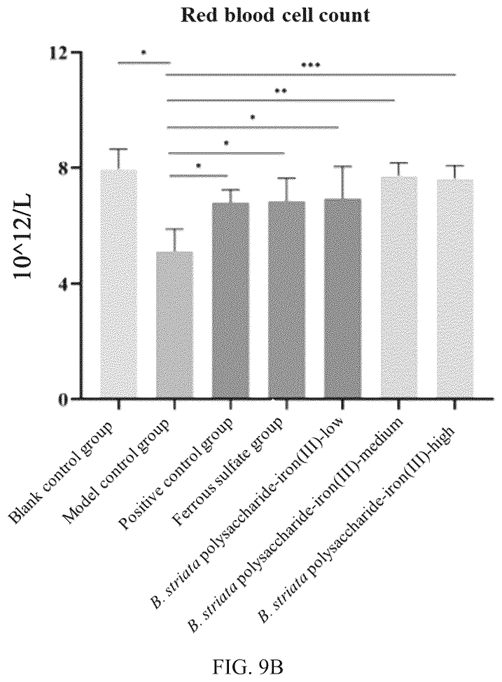

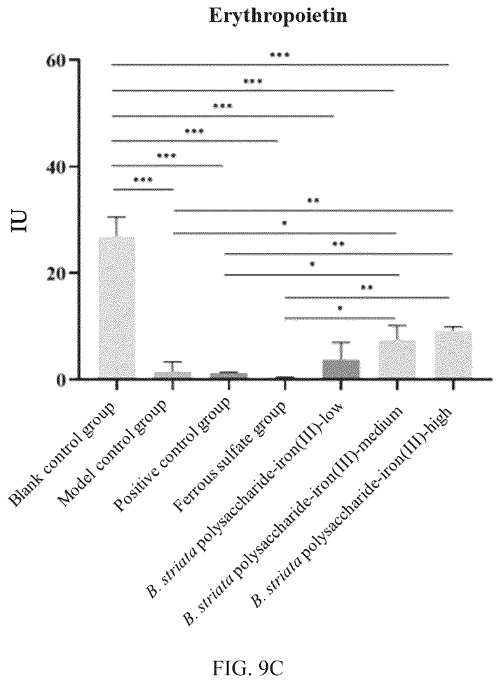

The Hb value can judge whether there is iron deficiency in the body and the survival of red blood cells, and is one of the main evaluation indexes of IDA. As can be seen from FIG. 9A, after 28 days of administration by gavage for treatment, the Hb values of the mice in the Bletilla striata polysaccharide-iron (III) complex low-dose, medium-dose and high-dose groups and the FeSO₄ group are not significantly different from that in the control group, which indicates that the above several drugs have a good effect on the recovery of red blood cells. However, the Hb value of the mice in the Niferex group is significantly different from that in the control group (0.01<p<0.05), which is possibly due to the fact that the Niferex molecules are larger, so that the absorption of Niferex in the body is not as good as that of FeSO₄ and the Bletilla striata polysaccharide-iron (III) complex. The Bletilla striata polysaccharide-iron (III) complex medium-dose and high-dose groups have extremely significant differences from the model group (p<0.01), with the Hb values being respectively 115.29 g/L, 112.99 g/L, and 73.67 g/L (n=5), and the other groups all have significant differences from the model group (0.01<p<0.05), indicating that the Bletilla striata polysaccharide-iron (III) complex medium-dose and high-dose groups have the best effects on the restoration of the Hb value of mice, and among the three drugs Niferex, FeSO₄, and Bletilla striata polysaccharide-iron (III) complex at the same dose, the Bletilla striata polysaccharide-iron (III) complex performs best. However, there is almost no significant difference in Hb value between the Bletilla striata polysaccharide-iron (III) complex medium-dose and high-dose groups, indicating that the absorption of the drug has little correlation with the concentration, which is consistent with the results of previous intestinal perfusion.

Red blood cells (RBCs) are the main cells in blood, accounting for 45% of the total blood amount, and RBCs are also an important indicator to reflect the condition of anemia in the body. As can be seen FIG. 9B, after 28 days of administration, none of the drugs shows difference values relative to the control group, and all of them have different significant differences from the model group. Among the groups, the Bletilla striata polysaccharide-iron (III) complex medium-dose and high-dose groups both have extremely significant differences from the model group (p<0.01), with the values being respectively 7.74 12/L, 7.64^{\hat{ }12}/L, and 5.12 10^{\hat{ }12}/L, indicating that the Bletilla striata polysaccharide-iron (III) complex medium-dose and high-dose groups have the best effect on increasing the RBC value and are superior to the Niferex and FeSO₄ groups (6.80^{\hat{ }12}/L and 6.85 10^{\hat{ }12}/L, respectively).

Erythropoietin (EPO) is a growth factor, and the amount of EPO can also reflect the normal proliferation of red blood cells in blood. As shown in FIG. 9C, although after 28 days of administration by gavage, there is still an extremely significant difference between the EPO value in the serum of each drug group and that in the control group, and there is also a certain difference in the restoration of EPO between each drug group and the model group. Among the groups, the Niferex group and the FeSO₄ group have almost no significant differences from the model group, indicating that the drugs for these two groups have no regulatory effect on EPO and cannot support its production, whereas the Bletilla striata polysaccharide-iron (III) complex medium-dose and high-dose groups show significant differences from the model group (0.01<p<0.05), with the values being respectively 7.64 IU, 9.26 IU, and 1.54 IU, and the high-dose group has a better ability to restore the EPO value in serum. This experiment demonstrates that the Bletilla striata polysaccharide-iron (III) complex cannot only increase the value of red blood cells in blood, but can also promote the production of red blood cells at the level of EPO.

The effect of the Bletilla striata polysaccharide-iron complex on the other hematological indexes of mice is as shown in Table 2.

TABLE 2

| Groups | HCT | MCV | MCH | MCHC | RDW |
|---|---|---|---|---|---|
| Control group | 0.42 ± 0.03 | 52.20 ± 1.76 | 18.43 ± 0.65 | 353.67 ± 4.04 | 18.63 ± 0.40 |
| Model group | 0.25 ± 0.07** | 44.20 ± 1.32** | 12.63 ± 2.43* | 284.00 ± 17.03 | 28.47 ± 2.14*** |
| Niferex group | 0.30 ± 0.02* | 50.51 ± 0.66 | 17.62 ± 0.84 | 400.66 ± 15.51 | 25.97 ± 1.86*** |
| FeSO₄ group | 0.29 ± 0.05* | 51.20 ± 2.07 | 17.83 ± 0.63 | 400.49 ± 16.03 | 25.56 ± 2.99** |
| Bletilla striata | 0.29 ± 0.06* | 51.08 ± 3.36 | 19.00 ± 2.30 | 428.49 ± 54.46 | 23.65 ± 1.02* |
| polysaccharide- | | | | | |
| iron(III) complex | | | | | |
| low-dose group | | | | | |
| Bletilla striata | 0.33 ± 0.07 | 51.61 ± 0.86 | 21.99 ± 4.60 | 489.33 ± 95.90*** | 23.12 ± 2.24* |
| polysaccharide- | | | | | |
| iron(III) complex | | | | | |
| medium-dose | | | | | |
| group | | | | | |
| Bletilla striata | 0.35 ± 0.03 | 51.98 ± 2.26 | 17.02 ± 2.23 | 377.78 ± 56.37 | 22.72 ± 2.74 |
| polysaccharide- | | | | | |
| iron(III) complex | | | | | |
| high-dose group | | | | | |

The hematocrit (HCT) represents the ratio of red blood cells to the total blood amount; the mean cell volume (MCV) refers to the mean volume of red blood cells, which reflects the mean size of red blood cells; the mean corpuscular hemoglobin (MCH) and mean corpuscular hemoglobin concentration (MCHC) reflects the Hb content in red blood cells; and the red blood cell distribution width (RDW) reflects the change in the heterogeneity of red blood cells. These indexes all indicate the basic situation of red blood cells in blood. Therefore, the anemia of the body can also be known by investigating these indexes. As can be seen from Table 2, the drug in each group have a strong ability to restore the above indexes. Among the groups, the Bletilla striata polysaccharide-iron (III) complex medium-dose and high-dose groups have the strongest ability to improve all the indexes, and there is no significant difference from the control group. In the RDW value measurement results, the Bletilla striata polysaccharide-iron (III) complex performs better in alleviating the heterogeneity of red blood cells than Niferex and FeSO₄, but still presents a significant difference from the control group (0.01<p<0.05). This is possibly due to the fact that IDA is just alleviated, and it is impossible to well produce a large number of red blood cells with normal morphology immediately; however, if the drug is taken for a prolonged time, the RDW value will tend to be normal.

(4) Effect of Bletilla striata Polysaccharide-Iron Complex on the Serum Iron Index of Mice

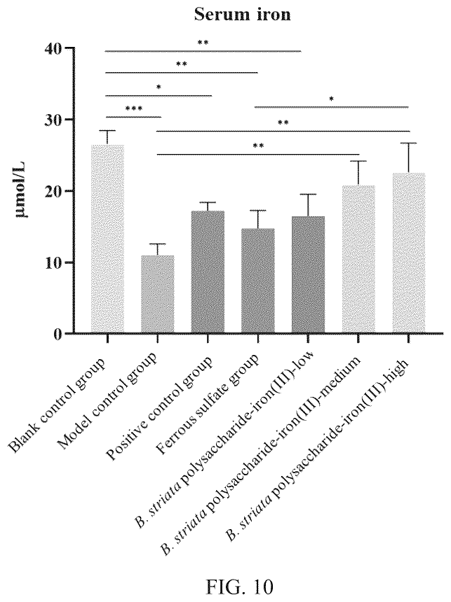

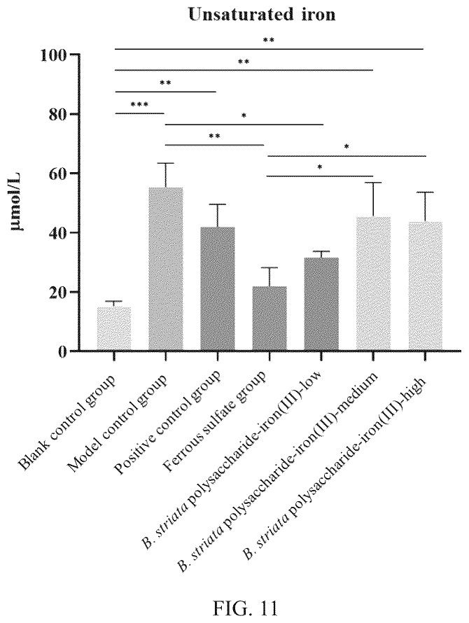

As can be seen from FIG. 10, the SI values of the mice in the Bletilla striata polysaccharide-iron (III) complex medium-dose and high-dose groups are close to that of the mice in the control group and have no significant difference, and the SI values of the mice in the high-dose group are significantly different from those in the FeSO₄ group (0.01<p<0.05), indicating that the Bletilla striata polysaccharide-iron (III) complex prepared by the present disclosure has a good iron supplementation ability against iron deficiency in the body. As can be seen from FIG. 11, all the drug groups can well reduce the TIBC of the body, indicating that the iron supplemented to the body by each of the drugs can be well absorbed, and the FeSO₄ group has the best effect, indicating that free iron is more easily utilized by hemoglobin or transferrin than bound iron. However, the Bletilla striata polysaccharide-iron (III) complex low-dose, medium-dose and high-dose groups also performed well and have no significant differences in effect from the Niferex group at the same dose.

(5) Effect of Bletilla striata Polysaccharide-Iron Complex on Organ Coefficients of Spleen and Liver in Mice

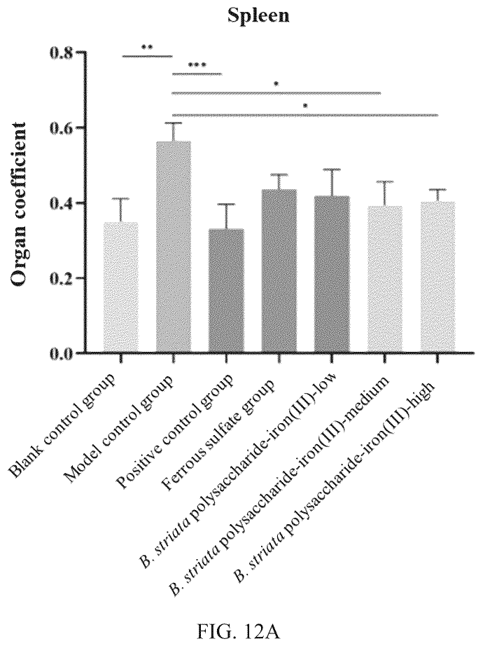

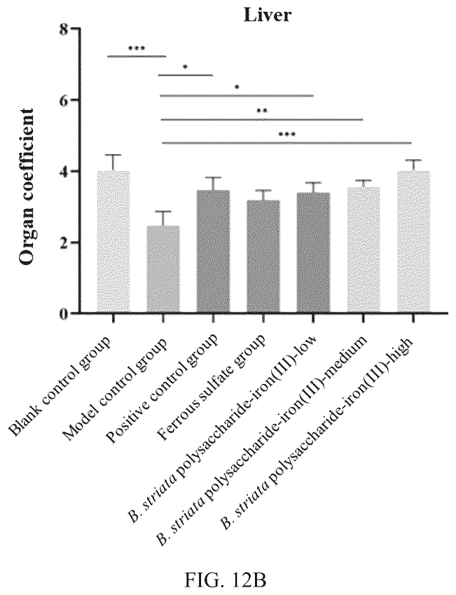

IDA can cause liver weight loss and spleen edema, and iron deficiency in the body will lead to the decrease of DNA synthesis and of the number of mitochondria in liver cells, thus inhibiting the growth of liver. In addition, the spleen can continuously repair hematopoietic tissues under the stimulation by IDA or some diseases, thereby causing splenomegaly. FIG. 12A and FIG. 12B show the organ index of mice in each group. As can be seen from FIG. 12A, the liver indexes of the mice in the model group and the mice in the control group are 2.48 and 4.04, respectively, with an extremely significant difference (p<0.01). It can be seen that the liver index of the mice in the model group is significantly lower than that of the control group, and this is caused by IDA. After 28 days of administration, the liver index of the mice in each drug group is increased to varying extent. Among the groups, the Bletilla striata polysaccharide-iron (III) complex medium-dose and high-dose groups all have extremely significant differences from the model group (p<0.01), and the Niferex group and the Bletilla striata polysaccharide-iron (III) complex low-dose group have significant differences from the model group (0.01<p<0.05); however, in the FeSO₄ group, the ability to restore the liver index is not strong. As can be seen from FIG. 12B, each drug group has a certain ability to alleviate splenomegaly, and the alleviating effects of the Bletilla striata polysaccharide-iron (III) complex medium-dose and high-dose groups have significant differences (p<0.01). The experimental results show that the Bletilla striata polysaccharide-iron (III) complex prepared by the present disclosure has a good effect on liver recovery and can alleviate organ damage and changes caused by IDA to a certain extent.

From the results of the above anti-iron deficiency anemia experiment in mice, it can be seen that the Bletilla striata polysaccharide-iron complex medium-dose and high-dose groups show better effects on the body weight recovery, the restoration of hematological indexes, the supplementation of serum iron, and the ability to alleviate pathologies in liver and spleen in mice with iron deficiency anemia than ferrous sulfate and the positive drug Niferex, indicating that at the same dose, the Bletilla striata polysaccharide-iron complex has a better therapeutic effect on iron deficiency anemia and can be used as a novel iron supplement for development and dosage form research.

Without departing from the concept of the present disclosure, several simple deductions or substitutions can be made by those of ordinary skill in the field to which the present disclosure belongs without involving any inventive effort. Therefore, according to the disclosure of the present invention, simple modifications made by those skilled in the art shall all fall within the scope of protection of the present disclosure. The above examples are some preferred examples of the present disclosure, and all processes similar to the present disclosure and equivalent changes made shall fall within the scope of protection of the present disclosure.

1. A method for preparing a Bletilla striata polysaccharide-iron complex, comprising the following steps:
(1) dissolving Bletilla striata polysaccharide and sodium citrate in distilled water, and under stirring condition, dropwise adding an NaHCO3 solution while heating to adjust a pH value of a reaction system; when a temperature of the reaction system rises to a reaction temperature, alternately dropwise adding the NaHCO3 solution and an FeCl3 solution to allow reaction until a red insoluble matter appears in the reaction system; and continuing the reaction to obtain a reaction solution; and
(2) subjecting the reaction solution to cooling, centrifugation, concentration, alcoholic precipitation, suction filtration, drying, redissolution, dialysis, and freezing to obtain the Bletilla striata polysaccharide-iron complex.

2. The method for preparing the Bletilla striata polysaccharide-iron complex according to claim 1, wherein in step (1), the pH value is 7.0-11.5, the reaction temperature is 50-90° C., and the reaction is continued for 0.5-3.0 hours.

3. The method for preparing the Bletilla striata polysaccharide-iron complex according to claim 1, wherein the Bletilla striata polysaccharide has a purity of 30%-99%, a mass ratio of the Bletilla striata polysaccharide to sodium citrate is (0.25-4): 1, and a mass-to-volume ratio of the Bletilla striata polysaccharide to the distilled water is (1-4) g: 100 mL.

4. The method for preparing the Bletilla striata polysaccharide-iron complex according to claim 1, wherein the NaHCO3 solution has a mass concentration of 5%-30%, and the FeCl3 solution has a molar concentration of 1-5 mol/L.

5. The method for preparing the Bletilla striata polysaccharide-iron complex according to claim 1, wherein in step (2), the cooling comprises cooling to room temperature under −80° C. to 4° C. condition; the centrifugation is conducted at a rotation speed of 1500-4000 r/min for 5-20 min; and the concentration comprises: collecting a supernatant resulting from the centrifugation, and subjecting the supernatant to rotary evaporation at 70-100° C. to ½-¼ of an original volume to obtain a concentrate.

6. The method for preparing the Bletilla striata polysaccharide-iron complex according to claim 5, wherein in step (2), the alcoholic precipitation comprises: adding an ethanol solution to the concentrate, stirring a resulting solution at 60-120 r/min for 3-5 min, and leaving the solution to stand at room temperature for 12-36 h, wherein the ethanol solution has a volume concentration of 50%-95%, and a volume ratio of the ethanol solution to the concentrate is (1-4): 1.

7. The method for preparing the Bletilla striata polysaccharide-iron complex according to claim 1, wherein in step (2), the drying is conducted at a temperature of 50-90° C. for 4-12 h, whereby a dried product is obtained; the redissolution comprises redissolving the dried product in distilled water, wherein a mass-to-volume ratio of the dried product to the distilled water is (1.5-10) g: 100 mL; and the dialysis is conducted by using a semi-permeable membrane with a molecular weight cut-off of 8000-14000 Da for desalting for 50-80 hours.

8. The method for preparing the Bletilla striata polysaccharide-iron complex according to claim 1, wherein in step (2), the freezing comprises: subjecting a solution after being subjected to dialysis to rotary evaporation at 70-100° C. to ½-⅕ of an original volume to obtain a rotary evaporation product, then prefreezing the rotary evaporation product at −20° C. to −80° C. for 1-7 days to obtain a prefreezed product, and then freeze-drying the prefreezed product in a vacuum for 1-5 days.

9. A Bletilla striata polysaccharide-iron complex prepared by the method for preparing the Bletilla striata polysaccharide-iron complex according to claim 1, wherein the Bletilla striata polysaccharide-iron complex has an iron content of 17.57±0.569%.

10. A Bletilla striata polysaccharide-iron complex prepared by the method for preparing the Bletilla striata polysaccharide-iron complex according to claim 2, wherein the Bletilla striata polysaccharide-iron complex has an iron content of 17.57±0.569%.

11. A Bletilla striata polysaccharide-iron complex prepared by the method for preparing the Bletilla striata polysaccharide-iron complex according to claim 3, wherein the Bletilla striata polysaccharide-iron complex has an iron content of 17.57±0.569%.

12. A Bletilla striata polysaccharide-iron complex prepared by the method for preparing the Bletilla striata polysaccharide-iron complex according to claim 4, wherein the Bletilla striata polysaccharide-iron complex has an iron content of 17.57±0.569%.

13. A Bletilla striata polysaccharide-iron complex prepared by the method for preparing the Bletilla striata polysaccharide-iron complex according to claim 5, wherein the Bletilla striata polysaccharide-iron complex has an iron content of 17.57±0.569%.

14. A Bletilla striata polysaccharide-iron complex prepared by the method for preparing the Bletilla striata polysaccharide-iron complex according to claim 6, wherein the Bletilla striata polysaccharide-iron complex has an iron content of 17.57±0.569%.

15. A Bletilla striata polysaccharide-iron complex prepared by the method for preparing the Bletilla striata polysaccharide-iron complex according to claim 7, wherein the Bletilla striata polysaccharide-iron complex has an iron content of 17.57±0.569%.

16. A Bletilla striata polysaccharide-iron complex prepared by the method for preparing the Bletilla striata polysaccharide-iron complex according to claim 8, wherein the Bletilla striata polysaccharide-iron complex has an iron content of 17.57±0.569%.

17. An iron supplement preparation comprising the Bletilla striata polysaccharide-iron complex according to claim 9.

18. An iron supplement preparation comprising the Bletilla striata polysaccharide-iron complex according to claim 10.

19. An iron supplement preparation comprising the Bletilla striata polysaccharide-iron complex according to claim 11.

20. An iron supplement preparation comprising the Bletilla striata polysaccharide-iron complex according to claim 12.
